(19) United States
(12) Patent Application Publication
(10) Pub. No.: US 2017/0106129 A1
(43) Pub. Date: Apr. 20, 2017

(54) BONDED MICROSPHERE FILTER

(71) Applicant: 1866402 ONTARIO LIMITED, Sudbury (CA)

(72) Inventors: Jeffrey SUTTON, Sudbury (CA); Angelo MAZZUCHIN, Sudbury (CA); Herman FALTER, Sudbury (CA); Michael STAFFEN, Sudbury (CA)

(21) Appl. No.: 15/311,077

(22) PCT Filed: May 14, 2015
(86) PCT No.: PCT/CA2015/000315

§ 371 Date: Nov. 14, 2016

Related U.S. Application Data
(60) Provisional application No. 61/996,740, filed on May 14, 2014.

(51) Int. Cl. A61M 1/02; B01D 39/20

(52) U.S. Cl. CPC A61M 1/0218; B01D 39/2013; B01D 39/2037; B01D 2239/0485; B01D 2239/1241; B01D 2239/125

(57) ABSTRACT

A filter material of microspheres bonded in a close-packed arrangement is provided. A filter device comprising a funnel containing a filter material of microspheres bonded in a close-packed arrangement is provided. The filter material and filter device are suitable for plasma separation from whole blood.

### CROSS-REFERENCE TO RELATED APPLICATIONS

This patent application claims priority to U.S. provisional patent application 61/996,740 filed May 14, 2014.

### TECHNICAL FIELD

The improvements generally relate to the field of microsphere filters.

### BACKGROUND OF THE ART

A particle or fluid filter is a device designed to physically block certain substances (in the feed) while letting others (the filtrate) through. Often, a filter is used to block oversized particles while passing fluid and undersized particles. Governing the particle size is the parameter Pore Size, which is the diameter of the holes in the filter or can be the diameter of the smallest particles retained in filter matrix—for material such as paper filters where filtering is caused by the bulk and density of woven fibers. Porosity is a volume ratio between the volume of empty space (void volume) and the total volume of the filter. Porosity is a key parameter in achieving higher flow rates through a filter. A sieve is a type of filter designed to separate at the surface of the sieve, not allowing larger particles to enter the material while a typical filter can allow oversize particles to enter the material but blocks the particles at some point in its thickness. In both cases filters and sieves are susceptible to blinding (blocking or clogging) whereby oversized particles coat the sieve or clog the filter in such a way as to prevent the filtrate from getting through, lowering or eliminating flow rate and total filtered volume.

In micro-fluidics, another important filter parameter is Hold-up Volume, the volume of filtrate retained in the filter after filtration. A low Hold-up Volume allows more filtrate to exit the filter, which is important for micro-fluidic processes. Hold-up Volume is generally related to void volume, and therefore also filter thickness. However, making filters thin typically has a negative effect on filtration, and on Wet Strength, a measure of how well a filter holds together under force of rupture. Particle retention efficiency is the ratio between the volume of oversized particles caught and the total volume of oversized particles in the feed. Recovery efficiency for a micro-fluidic filter can be thought of as a ratio of total volume of the actual filtrate exiting the filter and the total volume of fluid in the feed that should have passed thru the filter.

In Clinical Biochemistry many diagnostic tests performed in a laboratory use either blood serum or plasma as the various cellular components may interfere directly with the test methodology leading to inaccurate test results. (Serum is plasma without the clotting factor.) This requires that the plasma or serum be filtered or separated from the cellular components of whole blood.

Traditionally, plasma and serum have been separated from whole blood by centrifuging either before (in the case of plasma-whole blood collected in a heparinised vacutainer) or after clotting (for serum specimens-whole blood collected in a vacutainer and allowed to clot). Centrifugation is time consuming and requires skills and equipment that is not generally available outside laboratory environments.

There are two general types of membrane filters in use today. One type uses a fiber weave so as to make use of capillary force to transport fluid laterally along the fibers. In this process, larger sized cellular material travels more slowly, resulting in a plasma front forming at the leading edge of the fluid. This type of separation is currently not conducive to quantitative analysis as the effective plasma volume is small, caught up in the filter matrix, and may be difficult to measure. As well, fiber media can sometimes shear and lyse the cellular matter causing hemolysis and degrading the sample.

The other type uses fiber filters and membranes in vertical flow format, for example, using circular discs of small thickness fixed in a cylindrical container. Fluid, for example whole blood, is transported to one side of the filter and only the filtrate can travel through to the outlet. Often small pressures are used to force the plasma through the filter, although the use of pressure can result in haemolysis, which can occur at pressure near and above 40 mBar, or perforation of the filter or membrane, which, in turn can lead to invalid results. As above, shearing issues can also occur. Such filters are typically flat as they are difficult to manufacture in a small format with significant bends or corners. Based on its characteristics, such filters generally have a rating for the maximum volume of whole blood that can be applied to the surface area of the filter while remaining effective. This is because cellular material in the blood either comes to sit directly on the surface of the filter or traverses into the filter and becomes stuck. If there is too much oversized cellular matter, the filter will clog preventing further plasma flow through the filter. This problem can be mitigated by using discs with larger surface area, but this gives rise to increasing Hold-up Volume and therefore, less plasma available at the outlet side. Recovery efficiency for this type of filter is typically mediocre and it is caught up in the filter or adjoining collection pad, not a separate liquid volume

EP 0 544 450 relates to a composition of matter in which microspheres are covalently bonded to a solid substrate and, optionally, to each other.

Shim and Ahn (“Rapid On-Chip Blood/Plasma Separator Using Hetero-Packed Beads at the Inlet of a Microchannel”, 14 International Conference on Miniaturized Systems for Chemistry and Life Sciences, Oct. 3-7, 2010) disclose a whole blood/plasma separator fabricated by packing beads at an inlet of a microchannel, which due to faster movement of particles and fluid around obstacles can yield a plasma front.

Various weaknesses in the above methods have heretofore made them impractical for Lab-on-a-Chip (LOC) design and operation. The lack of a blood/plasma separator compatible with LOC has held up the commercialization of LOC.

### SUMMARY

In accordance with one aspect, there is provided a filter material comprising microspheres in a close packed arrangement bonded together so as to maintain interstitial holes open to fluid flow.

In one aspect, the microspheres have an average diameter of between about 1 nm and about 1000 μm. In another, the microspheres have an average diameter of between about 1 μm and about 100 μm. In another, the microspheres have an average diameter of between about 3 μm and about 20 μm.

At least a portion of the microspheres are suitably bonded together using chemical cross-linking, are laminated together and/or are bonded together using a magnetic force.

In one aspect, the microspheres of the filter material are cross-linked by covalent bonding.

In one aspect, the filter material is prepared by a process comprising coating microspheres with one or more a compounds comprising an epoxy or amine group and then reacting the coated microspheres with a compound comprising an azido and/or alkynyl group. Suitable compounds comprising an epoxy or amine group are PGMA and/or PEI. Compounds comprising an azido and/or alkyl groups include: 5-azidopentanoic acid, 4-ethynyl aniline, 4-pentyn-1-amine, 4-azidoaniline.hcl, 3-azido-1-propanamine, glycidyl propargyl ether and sulfosuucinimidyl-6-(4′-azido-2′-nitrophenylamino)hexonate (sulfo-SANPAH).

In one aspect, the filter material is prepared by coating a first portion of microspheres with azidopropyltriethoxysilane and a second portion of microspheres with O-(propargyl)-N-(triethoxysilylpropyl) carbamate and bringing the two portions of microspheres into contact with each other.

In one aspect the filter material is prepared by coating a first portion of microspheres with azidopropyltriethoxysilane and then reacting the first portion of the microspheres with glycidyl propargyl ether and coating a second portion of microspheres with GOPS and then reacting the second portion of microspheres with 3-azido-1-propanamine and bringing the two portions into contact with each other.

In one aspect, the filter material is prepared by coating a first portion of microspheres with at least one thiol or mercapto functional compound and coating a second portion of micropheres with at least one alkyne functional compound and bringing the two portion into contact with each other. Suitable mercapto functional compounds include 11-mercaptoundecyltrimethoxysilane. Suitable alkyne functional compounds include O-(propargyl)-N-(triethoxysilylpropyl)carbamate.

The filter material may be prepared by coating a first portion of the microspheres with (aminopropyl)triethoxysilane or 2,2-dimethoxy-1,6-diaza-2-silacyclooctane and a second portion of the microspheres with carboxyethylsilanetriol, sodium and bringing the two portions of microspheres into contact with each other. The microspheres may be bonded to each other by avidin or strepavidin and biotin. The microspheres may be bonded to each other using gluteraldehyde.

The filter material may comprise layers of bonded microspheres, at least two of the layers having different average diameters. In one embodiment, the filter material includes a plurality of layers of bonded microspheres arranged to form a gradient of increasing average diameter.

In one aspect, the microspheres comprise an organic polymeric material, which may be selected from polystyrene, polyaldehyde, polyacrolein, polyacrylic acid, polyglutaraldehyde and poly(methyl methacrylate) (PMMA). In another aspect, the microspheres are formed of an inorganic material, which may be selected from glass, silica and stainless steel.

In another aspect, there is provided a filter device for separating filtrate from a fluid comprising: a filter material comprising microspheres in a close packed arrangement and bonded together so as to maintain interstitial holes open to fluid flow; and a substrate for supporting the filter material.

In one aspect, the microspheres have an average diameter of between about 1 nm and about 1000 μm. In another, the microspheres have an average diameter of between about 1 μm and about 100 μm. In another, the microspheres have an average diameter of between about 3 μm and about 20 μm.

In one aspect, at least a portion of the microspheres are bonded together using chemical cross-linking. In one aspect, the microspheres are cross-linked by covalent bonding or are bonded to each other by avidin or streptavidin and biotin.

In one aspect, at least a portion of the microspheres are laminated together. In another, at least a portion of the microspheres are bonded together using a magnetic force.

In one aspect, the microspheres of the filter device comprise a polymeric material selected from polystyrene, polyaldehyde, polyacrolein, polyacrylic acid, polyglutaraldehyde and poly(methyl methacrylate) (PMMA) or an inorganic material selected from glass, silica or stainless steel.

In another aspect, the substrate comprises a material selected from polystyrene, silica, cellulose and stainless steel. A first layer of microspheres may be bonded to the substrate, such as by a magnetic force, chemical cross-linking or lamination.

The substrate may be shaped to form a fluid reservoir for receiving the fluid to be filtered, at least a portion of the fluid reservoir being coated with the filter material. The form may include an inlet for introducing fluid to the reservoir and an outlet for receiving filtrate that has passed through the filter material.

In one aspect, the device comprises layers of microspheres, at least two of the layers having a different average microsphere diameter. In one aspect, the filter material includes a plurality of layers of bonded microspheres arranged to form a gradient of increasing average diameter from the reservoir to the outlet.

In one aspect, the substrate is shaped into a funnel and the bonded microspheres are supported within the funnel.

The filter device may include an air inlet for introducing an air plug to separate a portion of filtrate collected in the outlet from the remainder of the filtrate.

In another aspect, there is provided a method for separating a filtrate from a fluid comprising: passing the fluid through microspheres bonded in a close-packed arrangement. In one aspect, the microspheres are bonded together using chemical cross-linking, magnetic forces and/or are laminated together.

In one aspect, the microspheres have an average diameter of between about 1 nm and about 1000 μm. In another, the microspheres have an average diameter of between about 1 μm and about 100 μm. In another, the microspheres have an average diameter of between about 3 μm and about 20 μm. In one aspect of the method, the fluid is passed through bonded microspheres of increasing average diameter.

In one aspect, the fluid is passed through at least one layer of bonded microspheres having an average diameter of between about 1 μm and about 20 μm. The fluid may be subsequently passed through at least one layer of bonded microspheres having an average diameter of between about 20 μm and about 50 μm; and, optionally, subsequently through at least one layer of cross-linked microspheres having an average diameter of between about 40 μm and 150 μm.

In one aspect, the method further includes applying a negative pressure of less than 40 mBar to draw the filtrate through the bonded microspheres.

In one aspect of the method, the fluid is whole blood and the filtrate is plasma.

In another aspect, there is provided a method of manufacturing a filter device comprising: a) assembling microspheres into a close-packed arrangement; and b) bonding the microspheres together to fix them in the close-packed arrangement without substantially blocking interstitial spaces between the microspheres. Steps (a) and (b) may be repeated to form a plurality of layers.

In one aspect, the microspheres are assembled into a close-packed arrangement by depositing the microspheres on a substrate and using compression, gravity, magnetic, or electrostatic force to assemble them into a close-packed arrangement.

In one aspect, at least a portion of the microspheres are bonded together using heat lamination, magnetic force and/or chemical cross-linking.

In one aspect, the microspheres and substrate are coated with a homobifunctional cross-linker. The substrate is suitably coated with a stoichiometric excess of the homobifunctional cross-linker.

In another aspect, a first portion of the microspheres is coated with a first cross-linker and a second portion of the microspheres is coated with a second cross-linker complementary to the first cross-linker and wherein the method comprises: depositing the first portion of microspheres on the substrate and assembling into a close-packed arrangement and binding the microspheres thereto; washing the substrate to remove unbound microspheres; depositing the second portion of microspheres on the substrate having the first portion of microspheres bound thereto and assembling into a close-packed arrangement and binding the microspheres thereto; and washing the substrate coated with the first portion and second portion of microspheres to remove unbound microspheres.

The method may further include curing the close packed arrangement of microspheres after washing of the substrate to remove unbound microspheres and may also include chemically reactivating cross-linked reagents after curing.

Many further features and combinations thereof concerning the present improvements will appear to those skilled in the art following a reading of the instant disclosure.

### DESCRIPTION OF THE DRAWINGS

FIG. 1 is a schematic illustration of close-packed spheres showing vias therethrough;

FIG. 2 is a schematic illustration of three sizes of close-packed spheres;

FIG. 3 illustrates a cross-linking reaction using carboxyethylsilanetriol, sodium (COS) and 2,2-dimethoxy-1,6-diaza-2-silacyclooctane (DMS).

FIG. 4 illustrates a coupling reaction of avidin or strepavidin to COS coated microspheres.

FIG. 5 is a schematic illustration of an embodiment of a filter device.

FIG. 6 is a schematic illustration of an embodiment of a filter device.

FIG. 7 illustrates the reaction chemistry of a glass substrate washed in a piranha solution (concentrated sulfuric acid H₂SO₄: 30% hydrogen peroxide H₂O₂ in a ratio of 7:3) rendering it hydrophilic.

FIG. 8 illustrates the reaction chemistry of a piranha solution treated glass substrate with a monolayer of DMS.

FIG. 9 illustrates the reaction chemistry of a DMS coated substrate with a monolayer of biotin.

FIG. 10 schematically illustrates the binding of strepavidin/avidin functionalized microspheres with biotin microspheres.

FIG. 11 illustrates the reaction chemistry between PGMA and propargylamine

FIG. 12 illustrates the reaction chemistry between PGMA and 3-azido-1-propanamine

FIG. 13 illustrates the reaction chemistry between PGMA-PEI and Glycidyl propargyl ether.

FIG. 14 illustrates the reaction chemistry between PGMA-PEI and sulfo-SANPAH

### DETAILED DESCRIPTION

In one embodiment, there is provided a filter material made by fixing microspheres in a close packed arrangement so as to maintain the interstitial holes between the microspheres so as to allow fluid flow therethrough. Suitable means of fixing the microspheres are chemical bonding, lamination, in particular heat lamination, and the application of magnetic force. Collectively, any means of fixing the microspheres in the close packed arrangement is referred to herein as bonding the microspheres. As will be clear to persons of skill in the art, certain bonding techniques will only be suitable for certain microsphere materials. A combination of bonding techniques may be used. Other means of fixing microspheres so as to maintain the interstitial holes may become apparent to persons of skill in the art and, in one embodiment, filter materials formed according to such methods are included within the scope of the present invention. The pore size of the filter, which is due to the interstitial holes, can only be maintained if the spheres stay packed, however, the present inventors have determined that in the absence of bonding, vibration, hydrophilic, or hydraulic forces can disturb the packing allowing larger diameter particles to flow through.

Sphere close packing is an overlapping arrangement of spheres in a containing space. It can be of equal size spheres but also of varying size ranges. Close packing is an arrangement that provides for the maximum density of spheres in the contained volume. The maximum possible close-packing, as stated by the Kepler Conjecture, is about 74% (Conway, J. H. and Sloane, N. J. A. Sphere Packings, Lattices, and Groups, 2nd ed. New York: Springer-Verlag, 1993.)

In order to provide an effective filter material, the microspheres are bonded so as to hold the spheres in their close-packed arrangement. The use of a bonding technique as described herein allows the microspheres to be held in a close packed arrangement, but without plugging the interstitial holes, which would happen using any typical glues such as organic (latex), solvent (Butanone), monomer (Cyanoacrylate), and polymer (Epoxy) based adhesives typically used for bonding microspheres.

When spheres are added to a constraining container and vibrated or compressed, they will generally form, under the force of gravity and the constraint of the container, what is known as random close-packing which is similar to maximum close-packing but with some irregularities. The density of this random close packing is believed to be never more than about 64% (Song, C.; Wang, P. & Makse, H. A. (29 May 2008). “A phase diagram for jammed matter”. Nature 453 (7195): 629-632) and therefore has a porosity of about 36% (F. A. L. Dullien, “Porous Media. Fluid Transport and Pore Structure”, 2nd edition, Academic Press Inc., 1992.). Magnetic and electrostatic forces can also be employed to cause close-packing. “Close packed” or “close-packing” are used herein to refer to an arrangement having a microsphere density of 50% to 74%. In one embodiment, between about 50% and about 64%. In various embodiments, the microsphere density is at least about 50%, at least about 55%, at least about 60%, at least about 65%, at least about 70%, and about 74%. As will be clear to a person of skill in the art, where a lower percentage of microspheres are fixed in close packed arrangement, it may be possible to nevertheless form an effective filter material through layering of the microspheres, and such layered arrangements also fall within the scope of embodiments of the present invention.

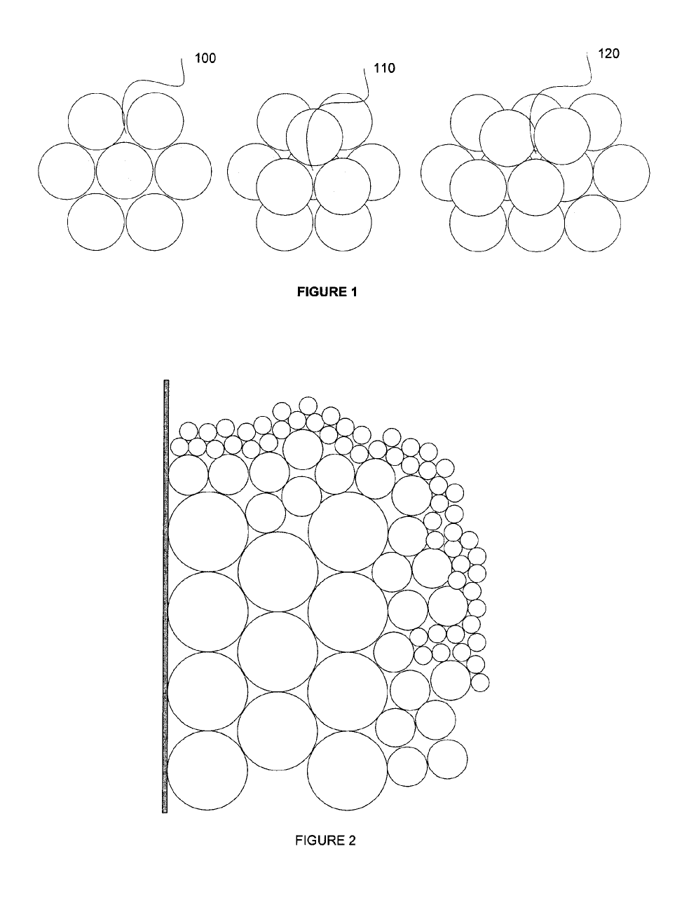

Packing leaves interstitial holes amongst the spheres and therefore paths, or vias, through the matrix as can be seen in FIG. 1. Such holes can be categorized as Trigonal holes 100, Tetrahedral holes 110, Octahedral holes 120, and Irregular (not shown). Close packing features Tetrahedral and Octahedral holes 110 and 120 with Trigonal holes 100 acting as the vias between all holes. Therefore the pore size of such matrix is based on the size of the Trigonal hole and the diameter of a particle that could traverse these vias. This diameter is directly related to the sphere diameter and is derived to be 0.15 times the diameter of the spheres used.

Irregular holes are those areas where spheres are not close packed and therefore gaps in the matrix are formed. To make use of an arrangement of spheres as a filter device, irregular holes are suitably minimized and, where existing, patched. FIG. 2 shows a segment where smaller spheres fill (patch) a gap or irregular hole.

Microspheres suitable for use in the present invention are small, spherical particles with diameters ranging from 1 nanometer up to 1 millimeter. In one embodiment, the microsphere is formed of any suitable material. Such microspheres can easily and cheaply be manufactured using known methods from various natural and synthetic materials and can be purchased from various sources, including e.g. Polysciences, Inc. The microspheres can be sorted or purchased according to diameter ranges. In one embodiment, the microspheres used in the filter material of the present invention have average diameters ranging from about 1 nm to about 1 mm. In one embodiment, the microspheres have an average diameter between about 1 μm and about 1 mm. In one embodiment, the spherical particles have an average diameter between about 1 μm and about 500 μm, about 400 μm, about 300 μm, about 200 μm, about 100 μm, about 20 μm or about 10 μm. In another embodiment, the spherical particles have an average diameter between about 3 μm and about 1 mm, about 500 μm, about 400 μm, about 300 μm, about 200 μm, about 100 μm, about 20 μm or about 10 μm. In another embodiment, the spherical particles have an average diameter between about 10 μm and about 1 mm, about 500 μm, about 400 μm, about 300 μm, about 200 μm, about 100 μm, or about 20 μm. In one embodiment, the microspheres have average diameter between about 1 nm and about 1 μm.

In one embodiment, the microspheres are formed of any suitable material such as would be within the purview of a person of skill in the art. In one embodiment, the microspheres are formed of organic polymeric material. In one embodiment, the microspheres are formed of organic polymeric material selected from polystyrene, polyaldehyde, polyacrolein, polyacrylic acid, polyglutaraldehyde and poly(methyl methacrylate) (PMMA). In one embodiment, the microspheres are formed of inorganic material. In one embodiment, the microspheres are formed of inorganic material selected from glass, silica and stainless steel.

In one embodiment, microspheres of different average diameter are used in the filter material. In one embodiment, microspheres of 2, 3, 4, or 5 different average diameters are used. In one embodiment, microspheres having more than 5 different average diameters are used.

In one embodiment of the filter material of the present invention, the porosity is not particularly restricted. In one embodiment, the porosity of the filter material is suitably between about 26% and about 50%, about 26% and about 45%, about 26% and about 40%, about 26% and about 35%, about 26% and about 30%, about 34% and about 50%, about 34% and about 45%, about 34% and about 40%. In one embodiment, the porosity of the filter material is about 37%. Pore size may suitably be controlled by choice of sphere diameter. Pore sizes can be fixed with specific values in the nanometer to micrometer range with specific tolerances. Material thickness can be finely controlled by using layering and thus thicknesses can be controlled with fine tolerances in the nanometer to micrometer range.

In another aspect, the present invention includes methods of making a filter material of microspheres bound together in a close-packed arrangement in such a way as to not significantly obstruct the interstitial holes naturally formed between the spheres. An excess of spheres is initially constrained in the close-packed arrangement, which can be done by placing them in a container of some geometry and using the force due to gravity, compression, magnetic, or electrostatic force to keep them in position. In one embodiment, the method used to bring the microspheres into close-packed arrangement is not particularly restricted.

Vibrating the container under the force of gravity will generally align the spheres in a random close packed arrangement. Irregular holes can be minimized by using a liquid slurry of individual microspheres and centrifuging or vibrating the slurry to achieve a close-packed matrix.

Electrostatic forces may be used to assemble microsphere material that can hold an electrostatic charge, such as polystyrene, into close-packed arrangement. The microspheres can be assembled on a substrate held at the opposite charge. The microspheres will self-assemble in a close packed arrangement due to electrostatic forces forcing them into the lower system energy state. McCarty, Logan S., Winkleman, A. and Whitesides, George M. (2007), Electrostatic Self-Assembly of Polystyrene Microspheres by Using Chemically Directed Contact Electrification. Angew. Chem. Int. Ed., 46: 206-209, the disclosure of which is incorporated herein by reference, reports a method for using electrostatic forces to hold and then bond polystyrene microspheres.

Ferromagnetic or paramagnetic microspheres will self-assemble into a close-packed arrangement with magnetic forces forcing the spheres into their lowest system energy state.

Once placed in the close-packed arrangement, the microspheres can be fixed in place (bonded) in such a way as to keep the naturally formed interstitial holes between the microspheres substantially clear and to prevent them from breaking apart under the pressures and flows associated with the filtration. In one embodiment, the microspheres can also be bonded to the constraining substrate. In another, the microspheres are not bonded to the substrate. Suitably, bonding can be from a continuing magnetic force, from heating the microspheres so as to laminate them together without substantially damaging their shape or changing the size of the interstitial holes, or by chemical bonding that does not substantially compromise the interstitial holes. A combination of bonding techniques may be used. For example, a hybrid of lamination and chemical bonding can be used whereby first layer(s) can be heat laminated to a substrate followed by washing and chemical techniques for additional layers.

As will be appreciated by a person of skill in the art, the methods of bonding described herein can be used both to bond microspheres to each other as well as to a supporting substrate, based on the selection of compatible materials and bonding methods.

Depending on the type of microsphere material used, one technique for bonding the microspheres is the use of heat to laminate or fuse the microspheres together in such a way that the spheres effectively maintain their spherical shape, but have fused with their neighbours. This method is particularly suitable e.g. for silica-based microspheres, an example of which is provided in Example 8.

In the case of ferromagnetic or paramagnetic microspheres, a continuing magnetic force may be used to fix the microspheres in close-packed arrangement. In one embodiment an electromagnet is placed under a small thin container made of glass, plastic, metal, or any material that will allow the magnetic field to pass, in such a way as to make a vertical and relatively constant magnetic field on the bottom surface of the container. Adding an excess of ferromagnetic or paramagnetic microspheres will result in a close packed arrangement of microspheres. This close packed arrangement may be fixed using a continuing magnetic field or, alternatively, another technique for bonding the microspheres may be employed.

In one embodiment, there is provided a method of layering the microspheres to provide a filter material.

In one method of chemical bonding, treated microspheres are formed in the desired arrangement by using a mechanical form and then bonded in-situ with a homobifunctional cross-link reagent. This method relies on the mechanical form to determine the geometry and material thickness. An example of such a method is provided in Example 6.

In another method of chemical bonding, at least two cross-linkers are used (here designated CL-A & CL-B) to build a filter device using heterobifunctional cross-linked layering. CL-A and CL-B are applied to sufficient quantities of the microspheres, which are then applied in alternating layers to a substrate. In one embodiment, one of CL-A and CL-B is capable of chemical cross-linking to the substrate. In one embodiment, the substrate is coated with CL-A or CL-B. In one embodiment, the substrate is shaped to form a fluid reservoir for receiving the fluid to be filtered and alternate filling and pouring off of the different treated microspheres is performed to deposit one layer at a time. Any irregular gaps and holes are generally patched by the alternating layers.

Bonding of the microspheres can be accomplished through the use of chemical silane coupling agents and cross-linking. As used herein, silane coupling agents are silicon-based molecules that have the ability to form a durable bond between inorganic and organic surfaces. A cross-link refers to a bond that links one polymer chain to another. In one embodiment, the filter materials are prepared using cross-link methods including heterobifunctional cross-linking, homobifunctional cross-linker, and Biotin-Avidin linking. With heterobifunctional cross-linking, microspheres are suitably treated with complementary silane coupling agent cross-linkers whereby they will readily bond to a microsphere coated with the complementary cross-linker but not to one with the same. In homobifunctional linking, microspheres are treated with the same chemical silane and a cross-linker chemical such as Gluteraldehyde is used to provide the linkage and bond.

Compared to filter paper and membranes, a filter made up of close-packed microspheres permits excellent porosity and enhanced control of pore size, thickness and matrix symmetry.

The use of chemical silanization and cross-linking to effectively coat the microspheres causes bonds to form wherever the microspheres come into contact. When reference is made to “coating” a substrate (e.g. microspheres or a supporting substrate) this generally refers to the application of a thin film of functional material to the substrate by methods known in the art or described herein (including for example, various methods of deposition, spraying, or dip coating). It will be understood that under certain circumstances, coating may include an incomplete application or the application of multiple layers or multiple applications of functional materials. Further, coating may be performed using e.g. a suitable solvent. Suitably, the chains of such cross-linkers are less than a nanometer long and do not plug the void spaces but do result in strong bonds. In such arrangements, each microsphere can be bonded to up to 3-12 other microspheres imparting strength to the matrix.

In one embodiment, the chemical cross-linker used in the filter material of the present invention is not restricted and any cross-linker known to those of skill in the art may be used. In addition to the chemical cross-linkers described below, other suitable cross-linkers will be apparent to those of skill in the art and certain suitable chemical cross-linkers may be obtained from commercial sources e.g. Gelest (PA, USA) and Pierce Protein Biology Products from Thermo Scientific (IL, USA).

In one embodiment, chemical cross-linking reactions performed to bond microspheres together and/or to bond microspheres to a supporting substrate may be performed using a suitable catalyst.

In one embodiment, silane cross-linkers with different functional groups attached are used to coat the surface of the microspheres, as shown schematically in the formula below:

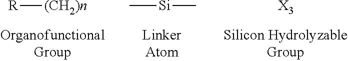

In one embodiment, organofunctional groups include chemical groups that are reactive towards primary amines, carboxyls, sulfhydryls, and carboxylic acids. Covalent and non-covalent binding occurs under established chemical conditions between the coated microspheres to create and bond layers of microspheres. Covalent bonding occurs between the functional groups such as the bond between the amino group of (aminopropyl)triethoxysilane (APTES) to the carboxyl group of carboxyethylsilantriol (COS). As used herein, the spacer arm length refers to the molecular span of a cross-linker (i.e., the distance between conjugated molecules). The surface contact area between spheres will vary depending on the length and structure of the cross-linker, and in one aspect, the spacer arm length is not particularly restricted and is limited only by the available chemical cross-linkers and may be selected to reflect the purpose for which the filter material is being prepared. However, as the C—C bond length is 1.54 Angstroms (0.154 nm), the length of cross-linkers are typically a few Angstroms and so will not clog the interstitial holes. As mentioned above, the pore size of the filter, which is due to the interstitial holes, can only be maintained if the spheres stay packed but, without bonding, vibration, hydrophilic, or hydraulic forces can disturb the packing allowing larger diameter particles to flow through.

In one aspect, azide and alkyne functional compounds and silanes can be used as cross-linkers to bond microspheres to each other and a supporting substrate through a reaction mechanism known as “1,3-dipolar cycloaddition” and also using “Click Chemistry” named and popularized by B. Sharpless—specifically a Copper-Catalyzed Azide-Alkyne Cycloaddition reaction mechanism.

In one embodiment, O-(propargyl)-N-(triethoxysilylpropyl) carbamate (YNE) and 3-azidopropyltriethoxysilane (AZ) silane compounds can be used to bond microspheres together and/or to bond microspheres to a supporting substrate. In one embodiment, after preparing a substrate with either a standard Piranha solution or Sodium Hydroxide (NAOH) treatment (both described in the Examples), the substrate components may be functionalized by a silane coating using 5% of the aforementioned silanes. After washing and annealing complementary functionalized substrate compounds may be bonded together using a solvent such as Hexane at room temperature for 1 hour.

The solvent used for the cycloaddition could be Tetrahydrofuran (THF), Dimethylformamide (DMF), Dimethyl sulfoxide (DMSO), Deionized water (DI) or combinations of solvents, e.g. such as 50% DI and 50% Tert butanol, which may be used at room temperature. Other suitable solvents for the cycloaddition reaction may be be known to those of skill in the art. The temperature at which the cycloaddition is performed may be optimized, e.g. in certain embodiments it may be performed at a temperature between room temperature and 120° C., in one embodiment between e.g 50° C. and 120° C., and a suitable temperature can be selected based on the solvent(s) used. Suitable reaction times can also be determined by those of skill in the art. In certain embodiments, the reaction time is between 1 and 24 hours or between 1 and 12 hours or between 4 and 8 hours.

In other embodiments, a copper catalyst is used to improve the reaction.

E.g. in a suitable reaction 0.2-2 mol % Copper(II) Sulphate (CuSo4.5H2O) plus 5-10 mol % of the reducing agent, Sodium Ascorbate may be added to a solvent such as 50% DI and 50% Tert Butanol at a temperature between room temperature and 120° C. for e.g. 50° C. for 1 hour to 12 hours e.g. 6 hours.

Other options for copper catalysts will be apparent to those of skill in the art. Suitable copper catalysts include e.g. Copper(1)Bromide plus a stabilizer such as Tris[(1-benzyl-1H-1,2,3-triazol-4-yl)methyl]amine (TBTA) or Triethylamine (TEA). In other embodiments, a phase transfer catalyst such as b-cyclodextrin may be used.

In other embodiments, cross-linker compounds may be linked to functional silanes such as epoxy or amine functional silanes. For example, cross-linker compounds such as 5-azidopentanoic acid, 4-ethynyl aniline, 4-pentyn-1-amine, 4-azidoaniline.hcl, and 3-azido-1-propanamine may be linked to amine and/or epoxy functional silanes and then bonded using the aforementioned 1,3-dipolar cycloaddition or Click Chemistry.

For example, (3-amixopropyl)triethoxysilane (APTES) functionalized components (amine/epoxy method described herein) may be reacted with a molar excess of glycidyl propargyl ether in a bicarbonate buffer (pH10) at room temperature for 12 hours to give propargyl functionalized components after washing. Glycidoxypropyltrimethoxysilane (GOPS) functional components may be reacted with a molar excess of 3-azido-1-propanamine in a bicarbonate buffer (pH10) at room temperature for 12 hours in dimmed light to give azide functionalized components after washing. These new functionalized components can be bonded as noted above. In this way similar cross linking with longer spacers can be employed to assist in increasing contact surface area and bonding.

In other embodiments, a base of poly(glycidyl methacrylate) (PGMA) along with polyethylenimine (PEI) may be functionalized with the noted crosslinker compounds and 1,3-dipolar cycloaddition or Click Chemistry may be used to bond these components.

For example a PEI functionalized component may be reacted with a molar excess of 5-azidopentanoic acid in a buffer of DMSO with 3% TEA at room temperature for 12 hours in dimmed light and then washed to produce an azide functionalized component. Other PEI components may be reacted with Glycidyl Propargyl ether as above. Again these complementary components may be bonded using 1,3-dipolar cycloaddition or Click Chemistry.

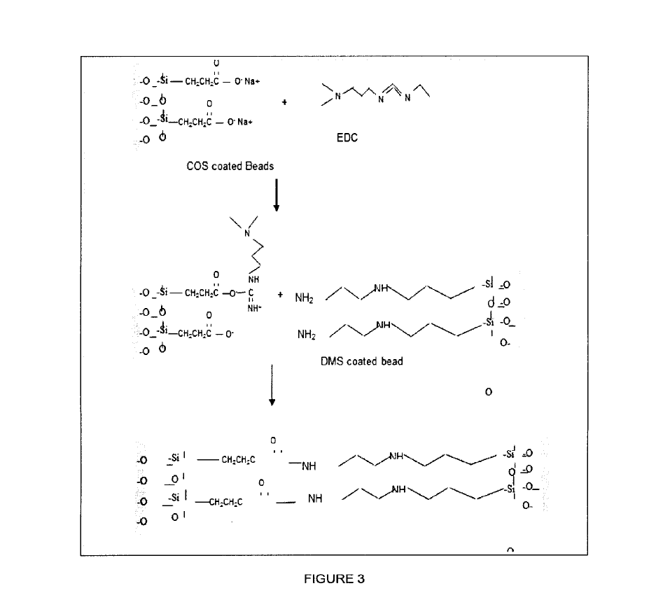

In one embodiment, cross-linking is achieved using carboxyethylsilanetriol, sodium (COS) and 2,2-dimethoxy-1,6-diaza-2-silacyclooctane (DMS). The chemical reaction is illustrated in FIG. 3. The thickness of a polyoxysiloxane layer is determined by the concentration of the oxysilane solution and a monolayer is generally desired. However, with some chemical cross-linking, multilayer adsorption often occurs, and these extra silane layers typically have weak bonding (hydrogen bonding, Van der Waals forces), and this polymerization can impede the desired bonding of microspheres directly onto a glass surface. In the reaction of DMS to COS, there is one primary amine for the carboxyl group to bind, which avoids polymerization. Further, the functional groups remaining after each of the layering can be re-activated by simple hydrolysis thus promoting the effectiveness of binding the next layer of spheres. A further advantage is that reactions can occur at one pH: Acidic (pH 4.5 to 5.5) MES buffer (4-morpholino-ethane-sulfonic acid) is effective, but phosphate buffers at pH≦7.2 are also compatible with the reaction chemistry. A further advantage is that reactions are fast, only taking a few minutes to complete

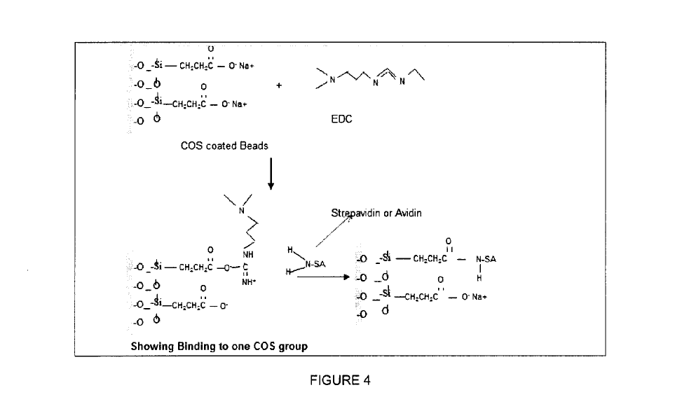

Avidin, a tetrametric protein, can bind with biotin with a dissociation constant of 10-15 M, which is one of the strongest known protein-ligand interactions. The four identical subunits of avidin can each bind one biotin. In one embodiment, Glycidoxypropyltrimethoxysilane (GOPS) coated microspheres are reacted with avidin at Alkaline pH to form GOPS-Avidin coated microspheres. In one embodiment, biotin is bound to DMS coated glass surfaces. The microspheres are then attached via the binding attraction between the avidin and biotin functional groups as illustrated in FIG. 4.

In one embodiment, layers of close-packed microspheres are assembled on a substrate and bonded. In one embodiment, the substrate has layers of microspheres, at least two of the layers having different average microsphere diameters. In one embodiment, the substrate has a plurality of layers of bonded microspheres layered thereon to form a gradient of increasing average diameter. In one embodiment, remaining holes can be patched by building multiple layers of spheres either by self-assembly layering or by use of a mechanical form.

In one embodiment, there is provided a filter device for separating filtrate from a fluid comprising a filter material as described above, comprising microspheres bonded in a close packed arrangement; and a substrate for supporting the filter material.

In one embodiment, the shape of a filter device according to the present invention is not particularly restricted and includes e.g. flat filters of any shape, cylindrical filters and cone-shaped filters.

In one embodiment, the filter is supported on a substrate having a geometric form and, in one embodiment, the shape of this form is not particularly restricted. In another embodiment, a filter device according to the present invention is formed on a cone-shaped geometric form.

In one embodiment, the substrate used can be formed of any suitable material, such as would be within the purview of a person of skill in the art. In one embodiment, the substrate is formed of polystyrene. In one embodiment, the substrate is formed of silica. In one embodiment, the substrate is formed of cellulose. In one embodiment, the substrate is formed of stainless steel. As will be understood by those of skill in the art, the substrate may need to be treated with a coating complimentary to the bonding agent.

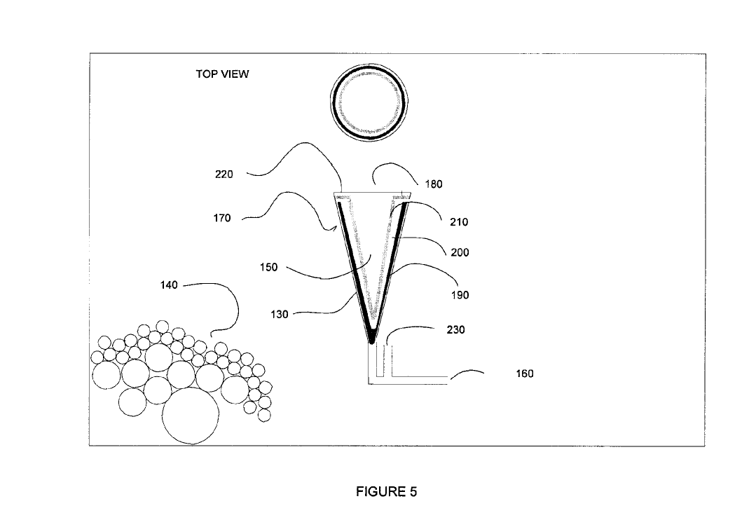

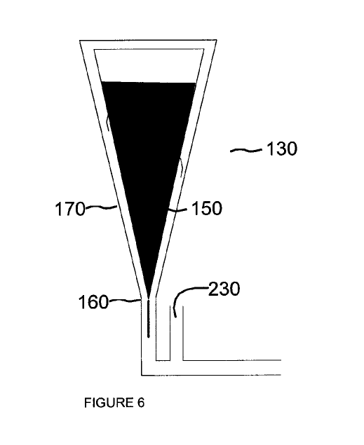

A filter device 130 employing the filter material described herein is described below with reference to FIGS. 5 and 6. This description is made with reference to the separation of plasma from whole blood, however, it is to be understood that the device can be used to separate any suitable filtrate from a fluid feed, the size of the microspheres being chosen as described above to yield the required Pore Size based on the desired filtrate.

In addition to separation of plasma from whole blood, filter materials and devices according to the present invention can be used in various diagnostic and other medical applications, including the separation of different cell types based on size, including red and white blood cells. Filter materials and devices of the present invention may also be used as particulate filters, for fluid (including air and water) purification, filtering of petroleum products, and particulate filtration and purification in the food and beverage industry (e.g. dairy processing). Various applications are possible by choosing the geometry, symmetry, and microsphere sizes (including nanometer diameters). Filter material and devices according to the present invention can also be used in various bio-processing applications and can be useful in sensors, including sensing contaminants in fluids, including air.

In whole blood, plasma makes up about 55% by volume meaning there is a significant amount of cellular matter to cause clogging. In filtration, it is important to maximize the ratio of surface area to volume (SA/V) so as to prevent clogging and speed up filtration. In geometry, the tetrahedron is the shape with the maximum SA/V, and a fluted funnel or cone is a structure with similar and significant SA/V. A fluted filter, or cone arrangement, according to an embodiment of the present invention, increases the speed of filtration by increasing the SA/V of the filter through which the solution seeps; and by allowing air to enter along its sides to permit faster pressure equalization. Accordingly, in one embodiment, the form is a cone wherein an outlet for the filtrate is provided at the point and the circular base of the cone in use provides an inlet for the fluid to be filtered. The form will be described herein as a funnel.

In the filter device according to this embodiment, any plasma in contact with the microsphere filter material or filter sides will readily flow into it while cellular matter being substantially larger than the designed pore diameter will not enter and, as it does not enter to any degree, it is readily able to move away from or along the material depending on other forces and dynamics.

As plasma seeps into the microsphere filter material 140, the volume of the solution in the sample reservoir 150 reduces; this reduction causes the remaining cellular matter and plasma to fall lower in reservoir 150 where due to the geometry of a cone, the surface area to volume ratio actually increases (SA:V=3 sin θ/h for a right circular cone) thereby bringing more plasma into contact with the surface. Some mixing will also occur bringing even more plasma into contact. The arrows in FIG. 6 illustrate plasma flow.

Further, in whole blood, cellular matter, being much denser than plasma, will naturally settle to the bottom of reservoir 150 due to gravimetric force. This settling effect gives rise to fluidic movement whereby the cellular matter drops, pushing plasma up and away. In the cone geometry with microspheres acting as a filter/sieve, and the walls being essentially vertical, at the wall interface, cells will tend to fall and not hold-up or clog the porous wall. As plasma is forced upwards it will gain unimpeded contact with the cone wall and readily flow into the porous walls.

Once in the microsphere filter material 140, plasma flows to the bottom capillary outlet 160 due to capillary forces aided by gravimetric forces and/or the application of a small negative pressure. As air can enter the system from the top edge of the cone and any uncovered top layers, this relieves any build-up of back pressure.

In a plasma separation device 130 according to one embodiment of the present invention, asymmetric microsphere material is bonded to the inner side of the funnel whereby the plasma flows into the sphere layers. A filter device 130 according to an embodiment of the present invention is shown in FIG. 5. Device 130 includes a shaped geometric form, funnel 170, the opening of which in use functions as an inlet 180 for whole blood to be separated. In use, plasma separated from the whole blood exits through outlet 160 at the base of funnel 170.

An inner surface of funnel 170 is coated with bonded microspheres as described herein. In the embodiment shown in FIG. 5, funnel 170 is coated with three layers of microspheres of different sizes, each layer individually comprising one or more layers of bonded microspheres. In the embodiment shown the layer proximate funnel 170, here designated the inner layer 190 comprises the largest diameter microspheres. In one embodiment, the microspheres of the inner layer have an average diameter of between about 20 μm and about 150 μm, about 140 μm, about 130 μm, about 120 μm, about 110 μm, about 100 μm, about 90 μm, about 80 μm, about 70 μm, about 60 μm, or about 50 μm; between about 30 μm and about 150 μm, about 140 μm, about 130 μm, about 120 μm, about 110 μm, about 100 μm, about 90 μm, about 80 μm, about 70 μm, about 60 μm, or about 50 μm; between about 40 μm and about 150 μm, about 140 μm, about 130 μm, about 120 μm, about 110 μm, about 100 μm, about 90 μm, about 80 μm, about 70 μm, about 60 μm, or about 50 μm; between about 50 μm and about 150 μm, about 140 μm, about 130 μm, about 120 μm, about 110 μm, about 100 μm, about 90 μm, about 80 μm, about 70 μm, or about 60 μm. In the embodiment shown, the intermediate layer 200 is formed of microspheres having an average diameter between that of inner layer 190 and outer layer 210. In one embodiment, the microsphere diameter of a layer is not so small as to fall into the interstitial holes of the adjacent layer. In another embodiment, it may be desirable that a percentage of microspheres enter the interstitial holes of the adjacent layer. In one embodiment, intermediate layer 200 is formed of microspheres having an average diameter of between about 10 μm and about 50 μm, about 40 μm, about 30 μm, or about 20 μm; between about 20 μm and about 50 μm, about 40 μm, or about 30 μm; between about 30 μm and about 50 μm or about 40 μm; between about 40 μm and 50 μm. In one embodiment, outer layer 210 is formed of microspheres having a smaller average diameter than inner layer 190 and intermediate layer 200. In one embodiment, outer layer 210 is formed of microspheres having an average diameter of between about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm 16 μm, 17 μm, 18 μm or 19 μm and about 20 μm In one embodiment, outer layer 210 is formed of microspheres having an average diameter of between about 2 μm and about 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm or 19 μm. In one embodiment, outer layer 210 is formed of microspheres having an average diameter of between about 3 μm and about 4 μm, 5 μm, 6 μm, 7 μm, 8 μm, 9 μm, 10 μm, 11 μm, 12 μm, 13 μm, 14 μm, 15 μm, 16 μm, 17 μm, 18 μm or 19 μm. In one embodiment, outer layer 210 is formed of microspheres having an average diameter of between about 4 μm and about 5 μm, 6 μm, 7 μm, 8 μm, 9 μm, 10 μm, 11 μm, 12 μm, 13 μm, 14 μm, 15 μm, 16 μm, 17 μm, 18 μm or 19 μm. In one embodiment, outer layer 210 is formed of microspheres having an average diameter of between about 5 μm and about 6 μm, 7 μm, 8 μm, 9 μm, 10 μm, 11 μm, 12 μm, 13 μm, 14 μm, 15 μm, 16 μm, 17 μm, 18 μm or 19 μm. Suitably the microspheres are such that the inner most layers are small enough to prevent cellular matter entering the interstitial holes while the outer most layers, the ones bonded to the inner side of the funnel 170, are larger to better enable fluid transport down the sides of the funnel 170 to the outlet 160 at the bottom. See FIGS. 2 and 5. When creating the microsphere layers, suitably, smaller layers are formed slightly higher, acting as a lip 220 over the outer larger spheres, to ensure whole blood does not enter the interstitial holes through the upper edge. See FIG. 5. Entry into the capillary should be overarched by the building of sphere layers, a configuration that can be facilitated by temporarily plugging the constriction with larger microspheres or glass fiber during manufacture of the filter device.

In one embodiment, there is further provided a method of separating plasma from whole blood using a filter device as described herein. Whole blood is added at the top of funnel 170 and spreads into the reservoir 150 coming into contact with the smallest diameter layer 210 on the sides of the funnel 170. In one embodiment, the microspheres are made hydrophilic. In one embodiment, a surfactant, which will lessen the surface tension of the fluid is used to enable fluid flow in and amongst the spheres. In one embodiment, an anticoagulant such as Heparin would be coated on the spheres to prevent coagulation and improve the overall flow of the fluid. As will be understood by person of skill in the art, depending on the uses to which filter devices of the present invention are put, surfactants, anticoagulants or other aids to filtration may be added to the sample to be filtered.

As the plasma leaks into the cross-linked microspheres, capillary forces and gravity and/or a small negative pressure will help propel the plasma down through the holes and vias to outlet 160 at the bottom (See FIG. 6). As well, as the whole blood volume in the reservoir 150 decreases and lowers down the sides of funnel 170, a dynamic flow in the whole blood is created that can enhance mixing and bring more plasma to the funnel sides and thus into the microsphere layers. This dynamic is continual until all available plasma has come into contact with the sides. The vertical sides of funnel 130, the smoothness of the microsphere surface, and small pore size tend to eliminate clogging as cellular matter will fall down the inner surface rather than get stuck in the entry area of the holes.

In one embodiment of the method, after a period of time from introduction of the whole blood into funnel 170, typically less than 5 minutes, a small vacuum pressure differential can be applied to the outlet 160 that will pull plasma still held up in the microspheres down to outlet 160. Although not restricted, in a preferred embodiment, the pressure applied is less than 40 mBar to limit haemolysis. Air replaces the plasma, coming in from the top of the sphere material, therefore little pressure differential is ever applied to the cellular matter. In one embodiment, the open end of the funnel 170 is covered by a cap (not shown). In one embodiment, the cap includes a fluid inlet for feeding fluid to be filtered into reservoir 150. In one embodiment, the cap includes an air inlet to allow air to flow in to replace any removed plasma volume. In one embodiment, the fluid inlet and the air inlet are the same inlet. In one embodiment, the cap is air permeable.

In one embodiment of the method, diluent is added to the whole blood, either prior to loading or as an additional component to the funnel. Finally, after receiving an acceptable volume of plasma, suitably an air plug is pumped into the capillary behind the plasma via air plug inlet 230 separating it from any subsequent leaking from the funnel.

In one embodiment, the device is a Lab-on-a-Chip technology (LOC). The design of LOC is to provide an integrated procedure from sampling to detection in a portable format so such testing can be performed at the point of care.

It will be apparent to those skilled in the art that various modifications and variations may be made in the materials, devices and methods disclosed herein without departing from the spirit and scope of the invention. It will be understood that elements of embodiments are not necessarily mutually exclusive, and many embodiments can suitably combined with other embodiments. For example, filtering devices may be manufactured using various combinations of microsphere materials, with various methods of bonding microspheres and in combination with supporting substrates of different materials, shapes and configurations.

### Example 1—Preparation of Glass Funnels

The stems of borosilicate glass pipettes (146 mm in length, 5 mm diameter at top) (Fisher Scientific) were cut 5 cm below the constriction of pipette (the capillary end) and 2 cm above the constriction. The top of a pipette was heated with a propane torch to soften and made to flare into a funnel until a diameter of about 7.5 mm was reached. A small layer of 50 diameter borosilicate glass microspheres was heat laminated just above the constriction point to act as a porous block to keep any added microspheres from falling out the bottom. While in this example, microspheres were used, other porous blocks can suitably be used and in other preparations glass wool (Fisher Scientific) has been used. The capillary end was heated and pinched to seal the bottom and contain reagents during sphere coatings. After all coatings were completed, the end was re-cut to open. The resulting cone had a height of 20 mm from the top to constriction, capable of holding a volume of 75-150 ul. The diameter of the top was 7.5 mm and the diameter of the constriction and outlet at the bottom was 1 mm.

### Example 2—Preparation of Glass Substrates

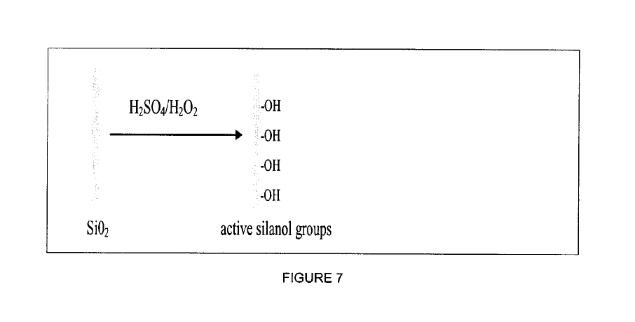

In this example, “glass substrates” refers to a funnel as prepared in Example 1 and borosilicate glass microspheres. In this example, glass microspheres of 3-10 μm, 10-30 μm and 30-50 μm from Polysciences, Inc. were used. The glass substrates were washed in a piranha solution (concentrated sulfuric acid H₂SO₄ (Fisher Scientific): 30% hydrogen peroxide H₂O₂ (Sigma Aldrich) in a ratio of 7:3) for 20 minutes with continuous stirring to clean the glass surface of organic matter and hydroxylate making it highly hydrophilic (see FIG. 7). The glass substrates were then thoroughly washed with distilled water and rinsed with iso-propanol (3 times) and then heated for 3 hrs at 110° C. The prepared glass substrates were stored in a desiccator until used.

### Example 3—Microsphere Layering of a Glass Funnel

In this Example, three sizes of microspheres were used: 3-10 μm, 20-30 μm, and 50-100 μm (borosilicate glass microspheres from Polysciences, Inc. prepared according to Example 2.). These sizes of microspheres yield a filter material particularly suitable for filtering plasma from blood (Red Blood Cells (RBC) 6-8 μm in diameter, White Blood Cells (WBC) 10-20, and Platelets 2-3 μm in diameter). The 3-10 μm spheres produce trigonal holes of in the 0.45-1.5 μm range, below the diameter of platelets. The 3-10 μm spheres may also fill some or all of the irregularities in the larger spheres which will have trigonal holes of 3-4.5 μm and 7.5-15 μm, respectively. In this Example, two layers of large spheres, four layers of the intermediate spheres and four layers of the smallest spheres were bonded. In this configuration the void volume is about 5 μl allowing for a good flow rate and a small Hold-up Volume.

Two cross-linkers were used (designated CL-A & CL-B, specific examples of which are provided in subsequent Examples) to build a filter material using heterobifunctional cross-linked layering. The funnel was coated with CL-A and quantities of microspheres were coated with CL-A & CL-B respectively. The funnel was first filled with the largest CL-B spheres in the form of a liquid slurry and gently vibrated (and, optionally, in certain protocols, centrifuged) to produce a close-packed arrangement. The coated funnel was then incubated for an appropriate time based on the type of reagents used. The funnel was then turned upside down and vibrated while flushing with buffer to empty non-bound spheres. Then the funnel with bound microspheres was cured according to the appropriate protocol based on the type of reagents used. The cross-link reagents were then reactivated as needed and per the reagents used.

Subsequent layers were added according to the same protocol. The addition of subsequent layers is preferably performed with vibration, but without centrifugation. Except for the last set of microspheres, suitably even numbers of layers are added for each size, as that way the first layer of a new size will be able to bond with the funnel surface. Suitably, sufficient microspheres are added in each new size to fill above the highest point of the previous layers so that a lip is created of smaller spheres. (See FIG. 5)

### Example 4—Preparation of Cross-Linked Glass Microspheres Filter Material Using Carboxyethylsilanetriol, sodium (COS) and 2,2-Dimethoxy-1,6-diaza-2-silacyclooctane (DMS)

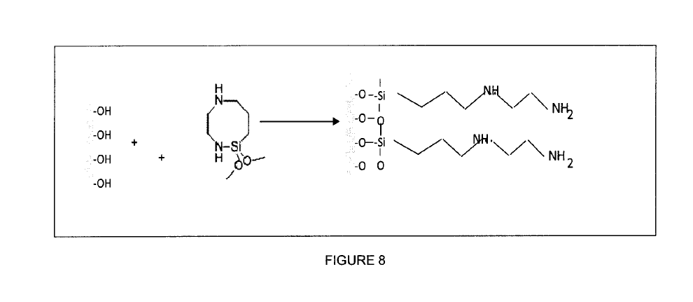

For the DMS coating protocol, glass funnels and microspheres as prepared in Example 2 are immersed in 5% (v/v) of DMS (Gelest Inc) in toluene at room temperature for 1.5 hrs, washed with toluene 3 times and cured at 110° C. for 3 hrs. The material is stored in a desiccator until used. The reaction chemistry for the formation of DMS monolayer is illustrated in FIG. 8.

For the COS coating protocol, glass funnels and spheres as prepared in Example 2 are immersed in 2.5% (v/v) of COS (Gelest Inc) in 95% ethanol at room temperature for 1.5 hrs, washed with ethanol 3 times and cured at 85° C. for 3 hrs. Material is stored in a desiccator until used. The reaction chemistry for the formation of COS monolayer can be seen with reference to FIG. 3 or 4.

A COS coated funnel is filled with a solution of 1-ethyl-3-[3-dimethylaminopropyl]carbodiimide hydrochloride (EDC) (Thermo Scientific) in phosphate buffer (10 mg EDC/ml of (buffer) for 5 minutes and then poured off. The funnel is then washed 3 times with phosphate buffer. Dried DMS spheres are added to just below the top of the funnel. The funnel is then gently vibrated and, preferably, for this step centrifuged. The microspheres are then covered with phosphate buffer. Vibration is then stopped and the funnel and contents are allowed to rest (a suitable rest time is about 10 minutes) and unbound microspheres and buffer are then poured off.

A slurry of COS spheres and EDC in phosphate buffer is prepared and allowed to incubate (a suitable incubation time is about 5 minutes) and then poured off. The microspheres are then washed 3 times with phosphate buffer. These microspheres are then poured into the funnel, covered with phosphate buffer and incubated (a suitable incubation time is about 10 minutes). The buffer is then poured off and the funnel is washed 3 times with buffer.

The process of coating with DMS and COS microspheres is repeated using alternating microspheres until the desired numbers of layers are completed.

### Example 5—Preparation of a Bonded Glass Microsphere Filter Material Using Binding of Biotin-Avidin/StrepAvidin

In the protocol for binding of StrepAvidin (SA), glass substrates coated with COS are immersed with a sufficient volume of SA (Sigma Aldrich) (1 mg/ml) and then mixed with 10 ul of EDC solution for each ml of protein. This mixture is then mixed on a shaker for 2 hrs. The microspheres are then washed extensively with Phosphate Buffer (PBS) (Fisher Scientific) (0.05% Tween™). The SA coated glass substrates are suitably stored dry in a desiccator until use.

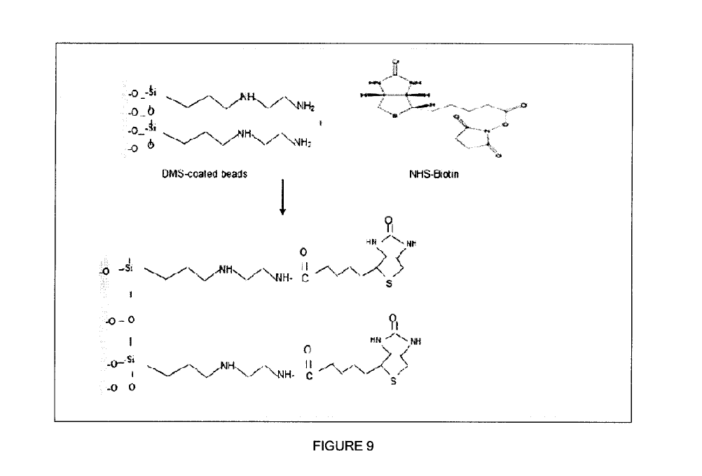

In the protocol for binding biotin (see FIG. 9) DMS coated spheres are immersed in NHS-BIOTIN solution for 1 hour [((+)-Biotin N-hydroxysuccinimide ester (Sigma Aldrich) dissolved in 99.9% DMSO to make up 10 mM solution (2 mg reagent in 590 ul of solvent)]. The microspheres are then washed extensively with PBS buffer and suitably are stored dry in a desiccator until used.

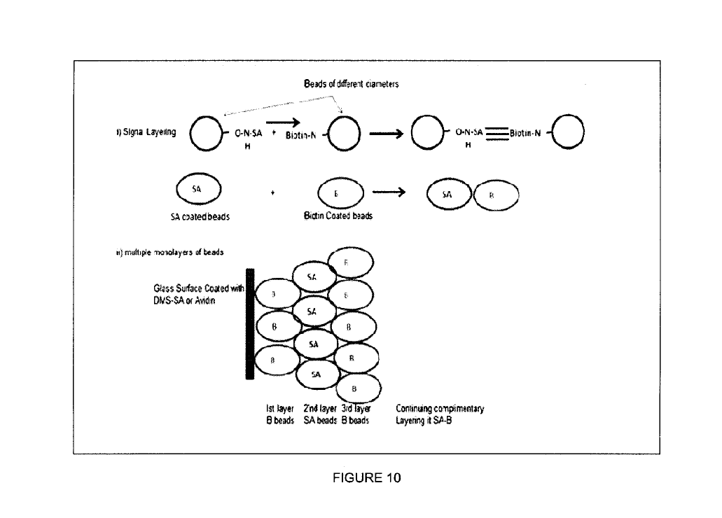

The Avidin and Biotin coated beads can then be layered as shown in FIG. 10.

### Example 6—Mechanical Forms Using Gluteraldehyde

DMS or APTES (available from Gelest) coated funnels were filled with DMS or APTES coated microspheres (10 to 30 μm). A sufficient volume of 8% glutaraldehyde (available from Thermo Scientific) (8% glutaraldehyde in PBS pH 7.4) was added to the funnel to cover all the microspheres. Pressure was applied by inserting a fitted inner cone of glass to compress the spheres in a uniform layer of microspheres, thus creating an inner cone of spheres attached to the walls of the funnel, which was then allowed to react at room temperature for 2 hours at room temperature. The inner cone was retrieved and the funnel was washed several times with PBS buffer (PBS buffer pH 7.5.) Following washing the gluteraldehyde-APTES bonds were made to be covalent by using the reducing agent Sodium Cyanoborohydride (available from Thermo Scientific) in a concentration of 10 mg/ml for 1 hour at room temperature. (Sodium Cyanoborohydride was used to reduce the shiff bases but leaving any remaining aldehydes active.)

### Example 7—Homobifunctional Cross-linking

Funnels and microspheres were Piranha-washed and dried overnight in an oven at 85 degrees Celsius. Funnel and microspheres were then immersed in 2.5% APTES mixed in 95% ethanol with 5% water for one hour. While immersed the microspheres were constantly mixed with a magnetic stirrer to prevent clumping. After 1 hour the funnels and microspheres were washed several times with ethanol and then allowed to cure for 4 hours in a vented oven at 85° Celsius. Then the APTES coated funnels were treated with 8% gluteraldehyde in PBS 8.5 for 1 hour at room temperature. This is done in large excess to minimize the occurrence of gluteraldehyde folding over and bonding to another site on the substrate. After one hour the funnels were washed several times with PBS 7.5 and then refilled with the reducing agent Sodium Cyanoborohydride (10 mg/ml) in PBS 7.5 for 1 hour at room temperature. Then the APTES microspheres were added to fill the funnel, and vibrated to close pack. PBS 8.5 was used to fill the funnel, taking care to remove air bubbles and wet all surfaces. After 1 hour the funnels were inverted and vibrated to remove any microspheres not bound. Cyanoborohydride is added with PBS 7.5 to reduce the bonds holding the microspheres to the funnel. This process creates a first layer of microspheres, however, as the APTES microspheres are still active on their unbound sides, the process can be repeated by again adding gluteraldehyde to create subsequent layers.

### Example 8—Lamination of Borosilicate Microspheres

In one embodiment, 30-50 μm diameter borosilicate glass microspheres were bonded to each other and the borosilicate glass funnel substrate that contained them in a random close packed arrangement. Adapting a technique from artistic glass works, the inner surface of the funnel was lightly coated with Elmer's white glue (Glue-All™), and left to dry until tacky. Then microspheres were poured into the funnel to excess while vibrating the funnel. Then a flat probe was used to compress the arrangement so that the outer edge of microspheres came in contact with the glass funnel through the lightly coated glue. After 10 minutes the funnel was inverted and vibrated to release any microspheres not glued to the inside surface of the funnel, while making sure that the coverage was complete. Then the funnel was placed in a Kiln and heated through a standard lamination protocol (heating to 1325 degrees Fahrenheit in 25 minutes, holding at this temperature for 15 minutes, dropping rapidly to 960 and then dropping back to room temperature slowly over 2 hours.) This procedure may be repeated to build up layers of random close packed microspheres adhered to a surface.

### Example 9—Using Alkyne and Azide Silanes with Click Chemistry

As in aforementioned examples microsphere and funnel substrates were piranha washed and dried.

Funnels and various sizes of microspheres were gently mixed in 5% 3-azidopropyltriethoxysilane (Gelest Inc.) (AZ) in 95% Etoh plus 5% water for 3 hours at room temperature and annealed for 3 hours at 110° C. Following the annealing the substrates were washed 3 times with Etoh. Other microspheres were gently mixed in 5% O-(propargyl)-N-(triethoxysilylpropyl) carbamate (Gelest Inc.) (YNE) in 95% Etoh plus 5% water for 3 hours at room temp and annealed for 3 hours at 110° C. Following the annealing the substrates were washed 3 times with Etoh.

Components were then wetted by sonication in hexane for 5 minutes and then the AZ funnel was packed with YNE microspheres and vibrated a few seconds to achieve close packing. Hexane was added to keep the components wet for the next hour and then dried for 1 hour at 60° C. for 3 hours. Unattached microspheres were then poured out. The next layer was achieved by repeating this process with the complimentary functioned microspheres.

This reaction can be made more efficient with the use of click chemistry, using a copper catalyst, changing solvents, and utilizing heat as mentioned previously using Click chemistry

### Example 10—Heterobifunctional Cross Linker Reactions of Alkyne/Azide Functionalized Glass Substrates

An alternative method to using silanes for the attachment of a surface monolayer to a substrate involves primary polymer (mono) layer with activity towards the substrate surface (glass) groups and with polymer functional groups available to react with other macromolecules.

In this example, a polymer layer of polyglycidyl methacrylate (PGMA) which binds to the activated hydroxyl groups on the glass substrate and provides functional surface epoxy groups was used as the base platform from which a variety of cross linker reactions were used to facilitate appropriate functionalized microsphere to substrate (here funnel) binding and sphere to sphere binding to create the filter membrane.

The building blocks consisted of funnels and microspheres coated with PGMA which provides epoxy functional groups at surface of the grafted polymer and PGMA-Polyethylenimine (PEI) coated funnels and microspheres which provide NH₂ functional groups at the surface. Heterobifunctional cross linkers were selected to bind to the epoxy functional groups in PGMA or primary amine functional groups on PGMA-PEI coated substrates.

Pre-treatment of glass substrates and microspheres: Funnels and spheres were sonicated in acetone for 10 min, treated in 0.5 N NaOH for one hour at 100° C., and washed three times in distilled water (DI). The funnels and microspheres were treated in DI for three hours at 100° C. for 1 hour and dried at 110° C. for 3 hrs.

### Example 10.a—Preparation of Polyglycidyl Methacrylate (PGMA Funnels and Spheres

Glass funnels and microspheres were immersed in 0.2% PGMA in methyl ethyl ketone (MEK) and mixed in a rotator for 3 hrs at 60° C. Substrates are washed with MEK once, decanted and annealed at 110° C. for 3 hrs. PGMA substrates were sonicated in ethanol for 5 minutes

### Example 10.b Preparation of PGMA-PEI funnels and spheres

PGMA coated funnels and microspheres were immersed in 2% polyethylenimine (PEI) solution of 20 mM solution of bicarbonate buffer at pH 10 and mixed in a rotator for 3 hrs at 60° C. After decanting liquid substrates were washed with ethanol and dried in oven at 60° C. PEI substrates were sonicated for 1 minute in ethanol.

### 10.c. Post Polymer Preparation of PGMA to Attach Alkyne and Azide Functional Groups

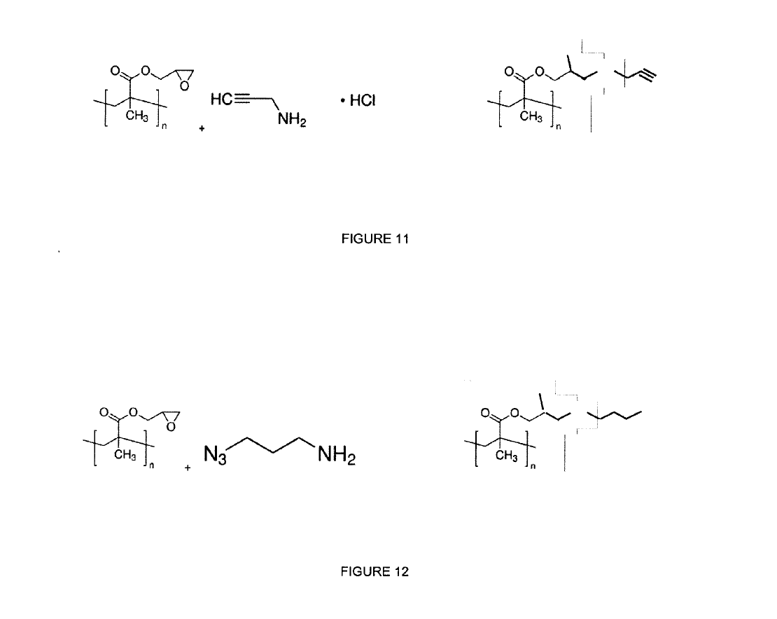

Examples of coupling agents that can be covalently bonded to the epoxy group in PGMA to result in alkyne functional groups that can later be reacted with azide groups via Click chemistry are propargylamine and 5-hexynoic acid The reaction with propargylamine is exemplified. PGMA coated funnels were reacted with 5% (v/v) propargylamine in 2-propanol for 6 hrs at 55° C. The reaction scheme with glass funnel is illustrated in FIG. 11 (Microspheres can be treated in the same manner)

PGMA Azide preparation with 3-azido-1-propanamine:

PGMA coated funnels were reacted with 4% (w/v) 3-azido-1-propanamine in THF for 3 hrs at room temperature. The reaction scheme with glass funnel is illustrated in FIG. 12. (Microspheres can be treated in the same manner)

### 10.d Post Polymer Preparation of PGMA-PEI to Attach Alkyne and Azide Functional Groups

PGMA-PEI Alkyne preparation with Glycidyl propargyl ether:

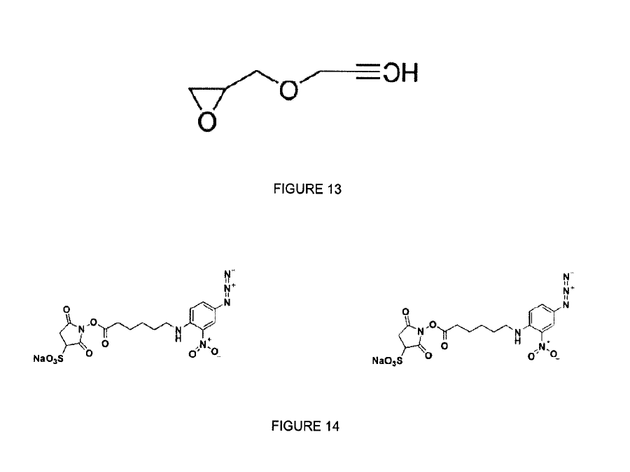

PGMA-PEI coated funnels are reacted with 16 mmol glycidyl propargyl ether in methanol overnight. The reaction scheme with glass funnel is illustrated in FIG. 13. (Microspheres can be treated in the same manner)

PGMA-PEI Azide preparation with sulfosuucinimidyl-6-(4′-azido-2′-nitrophenylamino)hexonate (sulfo-SANPAH):

PGMA-PEI coated funnels were reacted in dim lighting with 10 mM sulfosuucinimidyl-6-(4′-azido-2′-nitrophenylamino)hexonate (sulfo-SANPAH) in 20 mM sodium phosphate, 0.15 NaCL pH 8 at room temperature for 12 hrs. The reaction scheme with is illustrated in FIG. 14. (Microspheres can be treated in the same manner)

The examples described above and illustrated are intended to be exemplary only. The scope is indicated by the appended claims, which shall be understood to encompass all equivalents falling within the scope and spirit of the invention.

1. A filter material comprising microspheres in a close packed arrangement bonded together so as to maintain interstitial holes open to fluid flow.

2. The filter material of claim 1, wherein the microspheres have an average diameter of between about 1 nm and about 1000 μm.

3. The filter material of claim 2, wherein the microspheres have an average diameter of between about 1 μm and about 100 μm.

4. The filter material of claim 3, wherein the microspheres have an average diameter of between about 3 μm and about 20 μm.

5. The filter material of claim 2, wherein at least a portion of the microspheres are bonded together using chemical cross-linking.

6. The filter material of claim 2, wherein at least a portion of the microspheres are laminated together.

7. The filter material of claim 2, wherein at least a portion of the microspheres are bonded together using a magnetic force.

8. The filter material of claim 5, wherein the microspheres are cross-linked by covalent bonding.

9. The filter material of claim 5, wherein the filter material is prepared by a process comprising coating microspheres with one or more compounds comprising an epoxy or amine group and then reacting the coated microspheres with a compound comprising an azido and/or alkynyl group.

10. The filter material of claim 9, wherein the one or more compounds comprising an epoxy or amine group is PGMA and/or PEI.

11. The filter material of claim 9, wherein the compound comprising an azido and/or alkyl group is selected from: 5-azidopentanoic acid, 4-ethynyl aniline, 4-pentyn-1-amine, 4-azidoaniline.hcl, 3-azido-1-propanamine, glycidyl propargyl ether or sulfosuucinimidyl-6-(4′-azido-2′-nitrophenylamino)hexonate (sulfo-SANPAH).

12. The filter material of claim 5, wherein the filter material is prepared by a process comprising coating a first portion of microspheres with at least one thiol or mercapto functional compound and coating a second portion of microspheres with at least one alkyne functional compound and bringing the two portion into contact with each other.

13. The filter material of claim 12, wherein the at least one thiol or mercapto functional compound is 11-mercaptoundecyltrimethoxysilane and the at least one alkyne functional compound is O-(propargyl)-N-(triethoxysilylpropyl)carbamate.

14. The filter material of claim 5, wherein the filter material is prepared by a process comprising coating a first portion of the microspheres with azidopropyltriethoxysilane and a second portion of the microspheres with O-(propargyl)-N-(triethoxysilylpropyl) carbamate and bringing the two portions of microspheres into contact with each other.

15. The filter material of claim 5, wherein the filter material is prepared by a process comprising coating a first portion of the microspheres with azidopropyltriethoxysilane and then reacting the first portion of the microspheres with glycidyl propargyl ether and coating a second portion of the microspheres with GOPS and then reacting the second portion of microspheres with 3-azido-1-propanamine and bringing the two portions into contact with each other.

16. The filter material of claim 5, wherein the filter material is prepared by a process comprising coating a first portion of the microspheres with (aminopropyl)triethoxysilane or 2,2-dimethoxy-1,6-diaza-2-silacyclooctane and a second portion of the microspheres with carboxyethylsilanetriol, sodium and bringing the two portions of microspheres into contact with each other.

17. The filter material of claim 5, wherein the microspheres are bonded to each other by avidin or strepavidin and biotin.

18. The filter material of claim 5, wherein the microspheres are bonded to each other using gluteraldehyde.

19. The filter material of claim 1 comprising layers of bonded microspheres, at least two of the layers having different average diameters.

20. The filter material of claim 19 comprising a plurality of layers of bonded microspheres arranged to form a gradient of increasing average diameter.

21. The filter material of claim 1, wherein the microspheres comprise an organic polymeric material.

22. The filter material of claim 21, wherein the organic polymeric material is polystyrene, polyaldehyde, polyacrolein, polyacrylic acid, polyglutaraldehyde or poly(methyl methacrylate) (PMMA).

23. The filter material of claim 1, wherein the microspheres are formed of an inorganic material.

24. The filter material of claim 23, wherein the inorganic material is glass, silica or stainless steel.

25. A filter device for separating filtrate from a fluid comprising: the filter material of claim 1; and
a substrate for supporting the filter material.

26-40. (canceled)

41. The filter device of claim 25, wherein the substrate comprises a material selected from polystyrene, silica, cellulose or stainless steel.

42. The filter device of claim 25, wherein a first layer of microspheres is bonded to the substrate by a magnetic force, chemical cross-linking or lamination.

43. The filter device of claim 25, wherein the substrate is shaped to form a fluid reservoir for receiving the fluid to be filtered, at least a portion of the fluid reservoir being coated with the filter material.

44. The filter device of claim 43, wherein the form comprises an inlet for introducing fluid to the reservoir and an outlet for receiving filtrate that has passed through the filter material.

45. The filter device of claim 25, wherein the device comprises layers of microspheres, at least two of the layers having a different average microsphere diameter.

46. The filter device of claim 45, wherein the filter material comprises a plurality of layers of bonded microspheres arranged to form a gradient of increasing average diameter from the reservoir to the outlet.

47. The filter device of claim 46 wherein the substrate is shaped into a funnel and the bonded microspheres are supported within the funnel.

48. The filter device of claim 47 comprising an air inlet for introducing an air plug to separate a portion of filtrate collected in the outlet from the remainder of the filtrate.

49. A method for separating a filtrate from a fluid comprising: passing the fluid through the filter material according to claim 1.

50. The method of claim 49, wherein the microspheres are bonded together using chemical cross-linking, magnetic forces or are laminated together.

51. The method of claim 50, wherein the microspheres have an average diameter of between about 1 nm and about 1000 μm.

52. The method of claim 51, wherein the microspheres have an average diameter of between about 1 μm and about 100 μm.

53. The method of claim 52, wherein the microspheres have an average diameter of between about 3 μm and about 20 μm.

54. The method of claim 49, wherein the fluid is passed through bonded microspheres of increasing average diameter.

55. The method of claim 54, wherein the fluid is passed through at least one layer of bonded microspheres having an average diameter of between about 1 μm and about 20 μm.

56. The method of claim 55, wherein the fluid is subsequently passed through at least one layer of bonded microspheres having an average diameter of between about 20 μm and about 50 μm;
and, optionally, subsequently
through at least one layer of cross-linked microspheres having an average diameter of between about 40 μm and 150 μm.

57. The method of claim 49 further comprising applying a negative pressure of less than 40 mBar to draw the filtrate through the bonded microspheres.

58. The method of claim 49, wherein the fluid is whole blood and the filtrate is plasma.

59. A method of manufacturing a filter device comprising:
a) assembling microspheres into a close-packed arrangement; and
b) bonding the microspheres together to fix them in the close-packed arrangement without substantially blocking interstitial spaces between the microspheres.

60. The method of claim 59, wherein steps (a) and (b) are repeated to form a plurality of layers.

61. The method of claim 59, wherein the microspheres are assembled into a close-packed arrangement by depositing the microspheres on a substrate and using compression, gravity, magnetic, or electrostatic force to assemble them into a close-packed arrangement.

62. The method of claim 59, wherein at least a portion of the microspheres are bonded together using heat lamination.

63. The method of claim 59, wherein at least a portion of the microspheres are bonded together using a magnetic force.

64. The method of claim 59, wherein at least a portion of the microspheres are bonded using chemical cross-linking.

65. The method of claim 64, wherein the microspheres and substrate are coated with a homobifunctional cross-linker.

66. The method of claim 65, wherein the substrate is coated with a stoichiometric excess of the homobifunctional cross-linker.

67. The method of claim 64 wherein a first portion of the microspheres is coated with a first cross-linker and a second portion of the microspheres is coated with a second cross-linker complementary to the first cross-linker and wherein the method comprises:
depositing the first portion of microspheres on the substrate and assembling into a close-packed arrangement and binding the microspheres thereto;
washing the substrate to remove unbound microspheres;
depositing the second portion of microspheres on the substrate having the first portion of microspheres bound thereto and assembling into a close-packed arrangement and binding the microspheres thereto; and
washing the substrate coated with the first portion and second portion of microspheres to remove unbound microspheres.

68. The method of claim 67 further comprising curing the close packed arrangement of microspheres after washing of the substrate to remove unbound microspheres.

69. The method of claim 68, further comprising chemically reactivating cross-linked reagents after curing.

70. (canceled)
